(19) United States
(12) Patent Application Publication
(10) Pub. No.: US 2025/0242050 A1
(43) Pub. Date: Jul. 31, 2025

(54) COMPOSITION FOR TREATING GOUT CONTAINING CYCLODEXTRIN-CONJUGATED POLYMER NANO-DRUG AND METHOD FOR TREATING GOUT

(71) Applicants: The Industry & Academic Cooperation in Chungnam National University (IAC), Daejeon (KR); The General Hospital Corporation, Boston, MA (US)

(72) Inventors: Min Woong KANG, Daejeon (KR); Jin Hyun KIM, Daejeon (KR); Hak Soo CHOI, Iksan-si (KR)

(73) Assignees: The Industry & Academic Cooperation in Chungnam National University (IAC), Daejeon (KR); The General Hospital Corporation, Boston, MA (US)

(21) Appl. No.: 18/428,820

(22) Filed: Jan. 31, 2024

(51) Int. Cl. A61K 47/69; A61K 47/54; A61K 47/59; A61P 19/06

(52) U.S. Cl. CPC A61K 47/6951; A61K 47/545; A61K 47/59; A61P 19/06

(57) ABSTRACT

The present invention can provide a composition for treating gout or a method for treating gout, the composition containing a nano-drug in which a cyclodextrin moiety is conjugated to an amino group of a polymer, wherein a visible or near-infrared fluorophore is further conjugated to a carboxyl group of the polymer or the nano-drug further contains at least one gout therapeutic agent, which forms a complex together with the cyclodextrin moiety.

### TECHNICAL FIELD

The present invention relates to a pharmaceutical composition for treating gout and a method for treating gout, the pharmaceutical composition containing a nano-drug in which a cyclodextrin moiety is conjugated to an amino group of a polymer.

The present invention relates to a pharmaceutical composition for treating gout and a method for treating gout, the pharmaceutical composition containing a nano-drug in which a cyclodextrin moiety is conjugated to an amino group of a polymer, wherein the nano-drug contains an average of 1 to 32 cyclodextrin moieties and has an average molecular weight of about 8,000 to 40,000 g/mol.

The present invention relates to a pharmaceutical composition for treating gout and a method for treating gout, the pharmaceutical composition containing a nano-drug in which a cyclodextrin moiety is conjugated to an amino group of a polymer and a fluorophore is further conjugated to a carboxyl group of the polymer, wherein the fluorophore is selected from the group consisting of clinically approved visible fluorophores including fluorescein, Cy3, Cy5, Cy5.5, Cy7, Cy7.5, and oxazine, and near-infrared fluorophores under clinical trials including ZW800-1, ZW800-1C, ZW800-3C, ZW800-PEG, ZW700-1, indocyanine green (ICG), IRDye800-CW (CW800), OTL38, and other near-infrared fluorophores.

The present invention relates to a pharmaceutical composition for treating gout and a method for treating gout, the pharmaceutical composition containing β-cyclodextrin-conjugated ε-poly-L-lysine.

### BACKGROUND ART

Gout is an inflammatory disease triggered by the accumulation of monosodium urate (MSU) crystals within cartilage, tendons, and surrounding tissues of joints and subcutaneous tissues, which are formed from a high concentration of uric acid in the blood and joint fluids due to hyperuricemia where uric acid remains to have an increased concentration in the blood.

Uric acid is an insoluble nitrogen compound belonging to the purine family and is a final product of the protein metabolism. Uric acid is typically an acidic substance excreted into the urine, but when uric acid, without excreting into the urine, floats as monosodium urate (MSU) crystals in the blood vessel and accumulates in the joints or tissues, immune cells are recruited to the corresponding tissues to cause pain and inflammation. These cause joint inflammation to yield recurrent seizure accompanying severe pain, and the deposits of tophi resulting from monosodium urate (MSU) crystals cause the deformation and disability in the joint. Gout inflammation is caused by monosodium urate (MSU) crystals, which are needle-shaped crystals, derived from uric acid. Therefore, uric acid, which is the cause of monosodium urate (MSU) crystals, is considered a danger signal due to tissue damage.

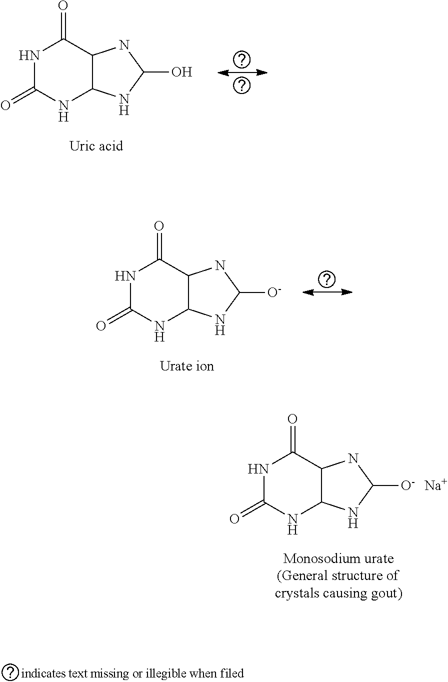 [SMILES: O=C1NC(=O)C2NC(O)NC2N1 ; O=C1NC(=O)C2NC([O-])NC2N1 ; O=C1NC(=O)C2NC([O-])NC2N1 ; [Na+]]

Neutrophils, which are a type of granular leukocytes mainly produced in the bone marrow, are cells of the same family as monocytes and are leukocytes which are the first to reach a site of the tissue when the tissue is damaged or infected with microorganisms. Several chemotactic factors, such as interleukin 8 (IL-8), are produced in the site, and a small peptide (formyl-methionyl-leucyl-phenylalanine) present in all the bacteria also acts as a powerful chemotactic factor to attract neutrophils, and these neutrophils play an important role in the intense acute inflammation. Therefore, in gout, neutrophils are most abundant in the inflammatory sites of gouty arthritis. The level of neutrophils is higher in joint aspirations of patients in pain.

The types of gout are broadly two: an overproduction type where uric acid itself is over-produced; and an underexcretion type where produced uric acid is not well excreted, with 90% of patients in South Korea being of the underexcretion type. Depending on the cause and type of gout, overproduction-type patients take medications with uric acid production inhibitors (e.g., allopurinol and febuxostat), and underexcretion-type patients take medications with uric acid excretion stimulators (e.g., probenecid, sulfinpyrazone, benzbromarone, and lesinurad). However, existing medications are only slightly effective or raise safety problems, such as side effects including allergies and urinary stones.

In 2012, the first choice uric acid-lowering agents recommended by the American College of Rheumatology (ACR) in the Guide to Gout Therapy were allopurinol and febuxostat, wherein febuxostat was first recommended as a first choice agent. Probenecid serves as a first choice agent to promote uric acid excretion in the uric acid-lowering therapy only when patients cannot take at least one xanthine oxidase inhibitor. In addition, the ACR guidelines recommended that patients start to receive uric acid-lowering therapy immediately after effective anti-inflammatory therapy begins. Therefore, there are still needs for new medications for the treatment or prevention of uratic or gouty diseases.

As for the prior art, Korean Patent Publication No. 10-2022-0016105 (published on 8 Feb. 2022) discloses a (3,5-dibromo-4-hydroxyphenyl) (6-hydroxy-2-(1-hydroxyethyl)benzofuran-3-yl-4,5,7-d3) methanone compound for the treatment and/or prevention of gout or hyperuricemia; Korean Patent No. 2187877 (registered on 1 Dec. 2020) discloses a mixed extract of Chrysanthemum indicum L. and Cornus officinalis Siebold εZucc. for the improvement, prevention, or treatment of hyperuricemia or gout; Chinese Patent Publication CN 114344483A (published on 15 Apr. 2022) discloses a targeted nano-drug for lung cancer treatment integration on the basis of an ultra-small molecule cyclodextrin-conjugated epsilon-poly-L-lysine nano-drug loading system and a method for producing the same; and PCT Publication No. WO 2018-005737A (published on 4 Jan. 2018) discloses an anticancer nano-carrier comprising at least one cyclodextrin (α-cyclodextrin, β-cyclodextrin, γ-cyclodextrin, 2-hydroxypropyl-β-cyclodextrin, 2-hydroxypropyl-γ-cyclodextrin, methyl-β-cyclodextrin, β-cyclodextrin thioether, or cyanoethylated β-cyclodextrin) nano-carrier conjugated to a polymer (polylysine, L-polylysine, polylactic acid, poly(lactic-co-glycolic acid), polyaspartic acid, polyglutamic acid, or polyglutamic acid-poly(ethylene glycol) copolymer), wherein a contrast agent is further included in the polymer of the nano-carrier and the cyclodextrin moiety and a therapeutic agent (anticancer agent) form a complex.

Nevertheless, there is still no gout therapy or medication that can achieve satisfactory effects.

### PRIOR ART DOCUMENTS

### Patent Documents

Korean Patent Publication No. 10-2022-0016105, Compound for the treatment of gout or hyperuricemia (publication date: 8 Feb. 2022)

Korean Patent No. 2187877, Composition containing as an active ingredient an extract of Chrysanthemum indicum L. and Cornus officinalis Siebold εZucc. for the improvement, prevention, or treatment of hyperuricemia or gout (registration date: 1 Dec. 2020)

Chinese Patent Publication CN 114344483A, Targeted nanodrug for lung cancer treatment integration on the basis of an ultra-small molecule cyclodextrin-conjugated epsilon-poly-L-lysine nanodrug loading system and method for producing the same (publication date: 15 Apr. 2022)

PCT Publication No. WO 2018-005737A, Renal clearable organic nanocarrier (publication date: 4 Jan. 2018)

### DISCLOSURE OF INVENTION

### Technical Problem

An aspect of the present invention is to provide a composition for preventing or treating gout and a method for treating gout, the composition containing a nano-drug in which a cyclodextrin moiety is conjugated to an amino group of a polymer.

### Solution to Problem

In accordance with an aspect of the present invention, there is provided a pharmaceutical composition containing a nano-drug in which a cyclodextrin moiety is conjugated to an amino group of a polymer.

In the present invention, the polymer provides a micelle, liposome, nano-sphere, dendrimer, or hollow shell, and examples of the polymer include ε-poly-L-lysine, L-polylysine, polylactic acid, poly(lactic-co-glycolic acid), polyaspartic acid, polyglutamic acid, or polyglutamic acid-poly(ethylene glycol) copolymers.

In the present invention, the cyclodextrin moiety is selected from α-cyclodextrin, β-cyclodextrin, γ-cyclodextrin, 2-hydroxypropyl-β-cyclodextrin, 2-hydroxypropyl-γ-cyclodextrin, methyl-β-cyclodextrin, β-cyclodextrin thioether, or cyanoethylated β-cyclodextrin, and at least one cyclodextrin moiety is conjugated to an amino group of the polymer.

In the present invention, the nano-drug further contains a contrast agent, wherein a fluorescent contrast agent is conjugated to the polymer. The contrast agent is selected from the group consisting of: clinically approved visible fluorophores including fluorescein, Cy3, Cy5, Cy5.5, Cy7, Cy7.5, and oxazine; and near-infrared fluorophores under clinical trials including ZW800-1, ZW800-1C, ZW800-3C, ZW800-PEG, ZW700-1, indocyanine green (ICG), IRDye800-CW (CW800), OTL38, and other near-infrared fluorophores.

The nano-drug contains at least one therapeutic agent, which forms a complex together with at least one cyclodextrin moiety.

In the present invention, the at least one therapeutic agent is a gout therapeutic agent, which is selected from nonsteroidal anti-inflammatory agents (aceclofenac, loxoprofen, meloxicam, lornoxicam, naproxen+esomeprazole, etodolac, nabumetone, indometacin, diclofenac, naproxen, zaltoprofen, dexibuprofen, pelubiprofen, ibuprofen, talniflumac acid, morniflumate, aspirin, ketoprofen, piroxicam, mefenamic acid, COX-2 selective inhibitor, celebrex, opioid receptor agonist, and tramadol), colchicine, adrenocortical hormone agents (hydrocortisone, dexamethasone, and methylprednisolone), uric acid-lowering agents, uric acid excretion stimulants (benzburomarone), or uric acid synthesis inhibitors (xanthine oxidase inhibitor, allopurinol, and febuxostat).

In the present invention, the nano-drug contains at least one positively charged moiety, and the nano-drug may contain about 1 to 32 positively charged moieties.

In the present invention, the nano-drug contains at least one negatively charged moiety, and the nano-drug may contain about 1 to 32 negatively charged moieties.

In the present invention, the nano-drug may contain at least one positively charged moiety and at least one negatively charged moiety, and the number of at least one positively charged moieties may be equal to the number of at least one negatively charged moieties.

In the present invention, the nano-drug contains about 1 to 32 positively charged moieties and about 1 to 32 negatively charged moieties.

The nano-drug may have an overall positive charge, an overall negative charge, or no charged moiety.

In the present invention, the nano-drug has an average molecular weight of about 8,000 to 40,000 g/mol.

In the present invention, the nano-drug may contain about 1 to 32 cyclodextrin moieties on average, preferably about 4 to 8 cyclodextrin moieties on average, about 8 to 12 cyclodextrin moieties on average, about 12 to 16 cyclodextrin moieties on average, or about 16 to 20 cyclodextrin moieties on average.

In the present invention, the nano-drug may have an average hydrodynamic diameter of about 1 to 10 nm.

For the nano-drug in which a cyclodextrin moiety is conjugated to an amino group of a polymer and a preparation method therefor according to the present invention, refer to preparation methods disclosed in PCT Publication No. WO 2018-005737A.

In the above preparation of nano-drugs, cyclodextrin (CD) is first converted to monoaldehyde-cyclodextrin (Ald-CD), and Ald-CD binds to an amino group of ε-poly-L-lysine (EPL) to which a visible or near-infrared fluorophore is bound, to form cyclodextrin-bound ε-poly-L-lysine (CDPL). Thereafter, the fluorophore-CDPL is succinylated to obtain zwitterionically charged fluorophore-CDPL^{±}.

As shown in <Scheme 1> below, for the conversion of cyclodextrin (CD) to Ald-CD, CD is dissolved in anhydrous dimethyl sulfoxide (DMSO), and dimethyl phthalate (DMP) is added to the resultant solution, followed by stirring at room temperature, and then the reaction solution is poured into an ethyl acetate/acetone mixture, followed by precipitation and then storage overnight. The next day, the precipitate is collected by vacuum filtration and dissolved in DMSO, and then this solution is poured into ethyl acetate/acetone, followed by centrifugation, and the supernatant is discarded. The precipitate is dissolved in deionized water and again poured into ethyl acetate/acetone to form a precipitate. After the precipitation, the precipitate is dissolved in deionized water, stirred, and then freeze-dried to collect a white solid. The white solid is monoaldehyde-cyclodextrin (Ald-CD).

In addition, for the binding of Ald-CD and ε-poly-L-lysine (EPL), Ald-CD is dissolved in an acetic acid buffer, and then EPL is added thereto and stirred at room temperature to obtain a mixture. Sodium borohydride is added to the mixture and continuously stirred at room temperature, followed by dynamic dialysis using a cellulose film with a molecular weight cut-off value of 6-8 kDa. After the dialysis, the resultant material is freeze-dried at −80° C. to produce an off-white solid, cyclodextrin 8-poly-L-lysine (CDPL).

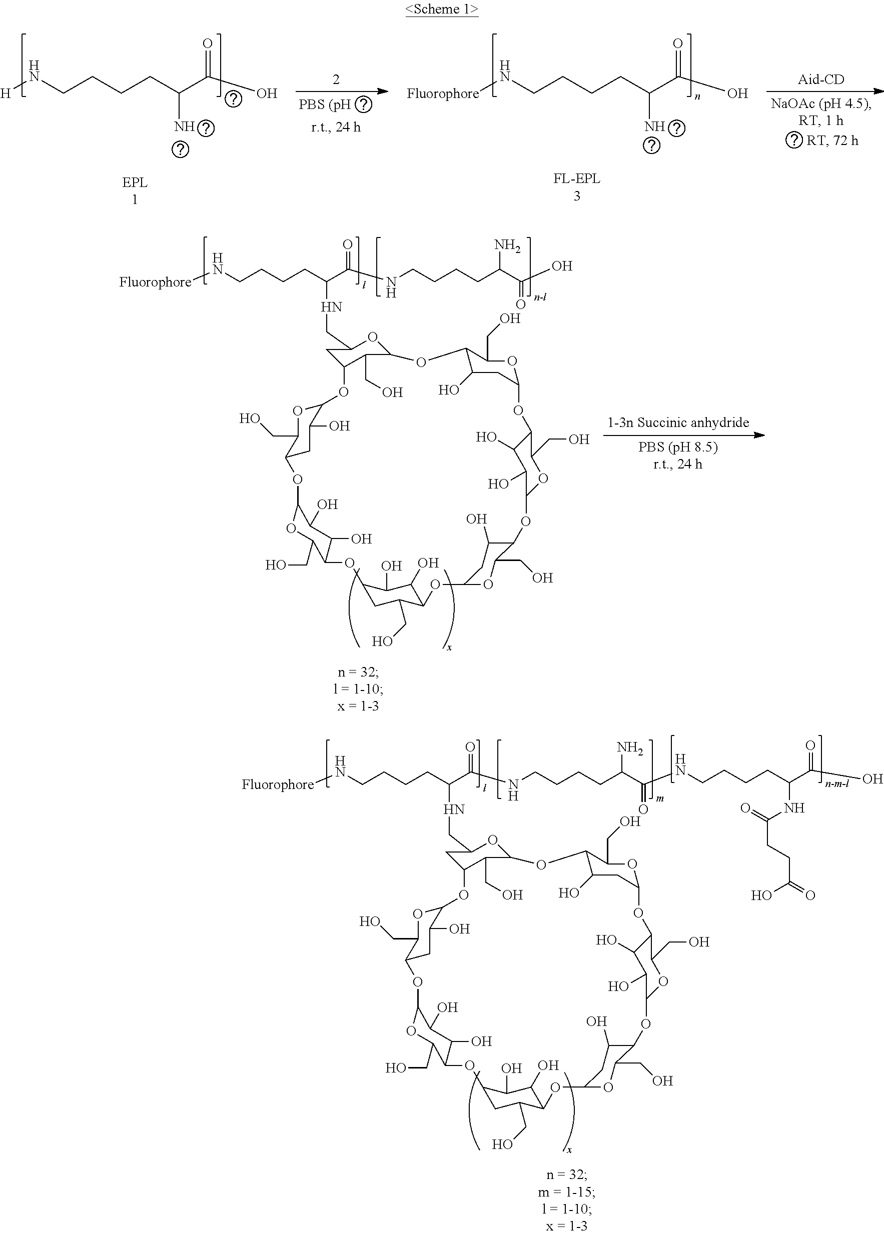

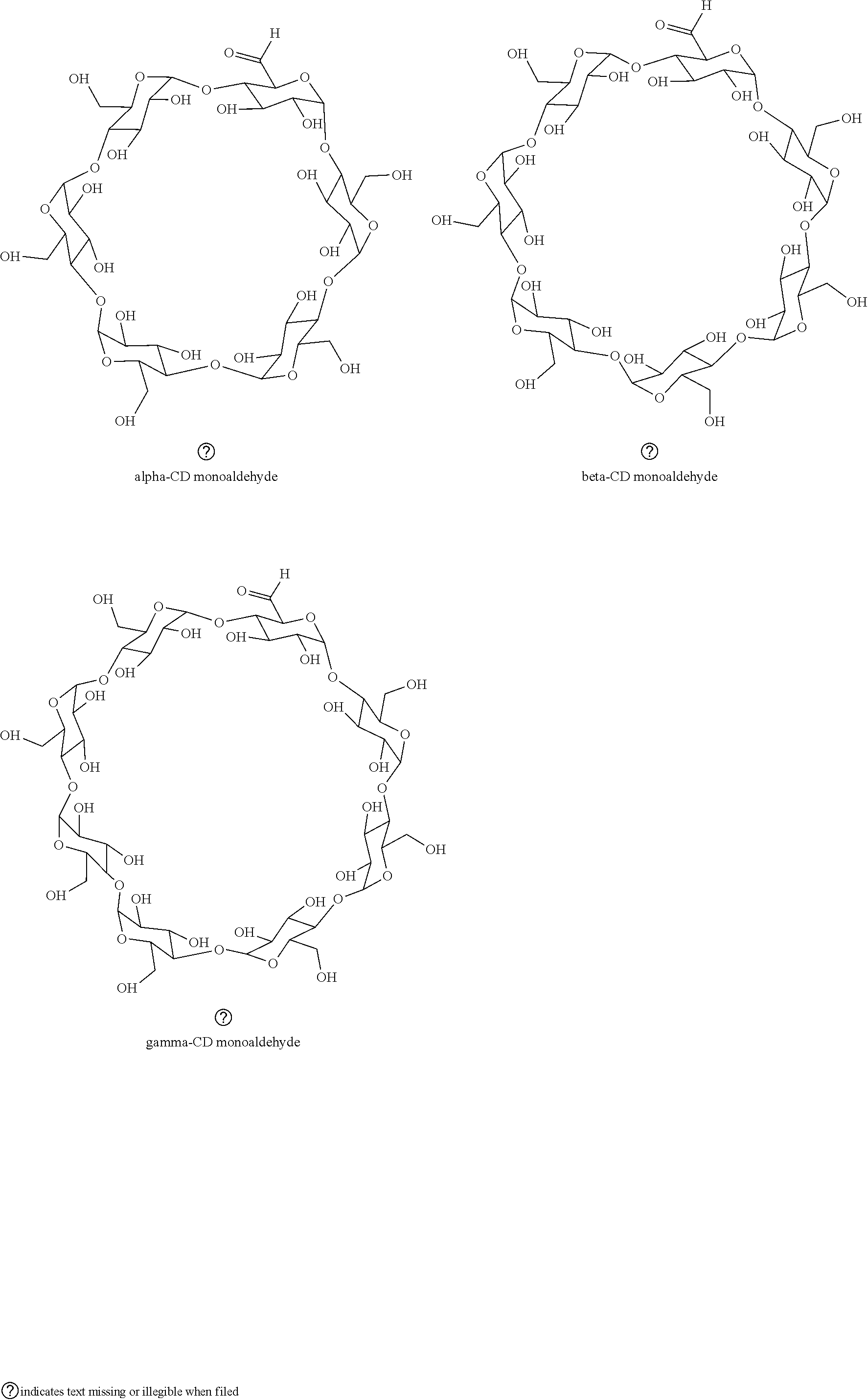 [SMILES: O=CC1OC2OC3C(CO)OC(OC4C(CO)OC(OC5C(CO)OC(OC6C(CO)OC(OC7C(CO)OC(OC1C(O)C2O)C(O)C7O)C(O)C6O)C(O)C5O)C(O)C4O)C(O)C3O ; O=CC1OC2OC3C(CO)OC(OC4C(CO)OC(OC5C(CO)OC(OC6C(CO)OC(OC7C(CO)OC(OC8C(CO)OC(OC1C(O)C2O)C(O)C8O)C(O)C7O)C(O)C6O)C(O)C5O)C(O)C4O)C(O)C3O ; O=CC1OC2OC3C(CO)OC(OC4C(CO)OC(OC5C(CO)OC(OC6C(CO)OC(OC7C(CO)OC(OC8C(CO)OC(OC9C(CO)OC(OC1C(O)C2O)C(O)C9O)C(O)C8O)C(O)C7O)C(O)C6O)C(O)C5O)C(O)C4O)C(O)C3O]

For the binding of the CDPL and the fluorophore, the fluorophore is dissolved in DMSO to prepare a fluorophore solution, and CDPL is dissolved in a phosphate buffer solution (PBS) to prepare a CDPL solution. Under stirring conditions, the fluorophore solution is dropped in the CDPL solution, followed by continuous stirring at room temperature. Thereafter, a sodium hydroxide (NaOH) aqueous solution is added to maintain the pH of the reaction solution to 8.0, and after three hours, the reaction solution is poured into ethyl acetate/acetone to precipitate a precipitate, followed by centrifugation. The supernatant is discarded, and the precipitate is again dissolved in deionized water and then again poured into ethyl acetate/acetone to obtain a precipitate, which is then vacuum dried to obtain solid fluorophore-CDPL.

The fluorophore is activated ZW800-1-NHS ester, ZW800-1C-NHS ester, or ZW800-PEG-NHS ester.

In addition, the succinylation of the fluorophore-CDPL is carried out as below. The succinylation is performed using a solution of dimethyl sulfoxide of succinic anhydride.

In the present invention, β-cyclodextrin (β-CD) is converted to monoaldehyde-cyclodextrin (Ald-CD), and Ald-CD binds to epsilon-poly-L-lysine (ε-poly-L-lysine, EPL) to which visible or near-infrared fluorophore is bound, to form a cyclodextrin-conjugated EPL (CD-conjugated EPL, CDPL). The cyclodextrin-conjugated EPL (CDPL⁺) undergoes fluorophore binding and is again succinylated, thereby preparing a fluorophore-CDPL^{±}.

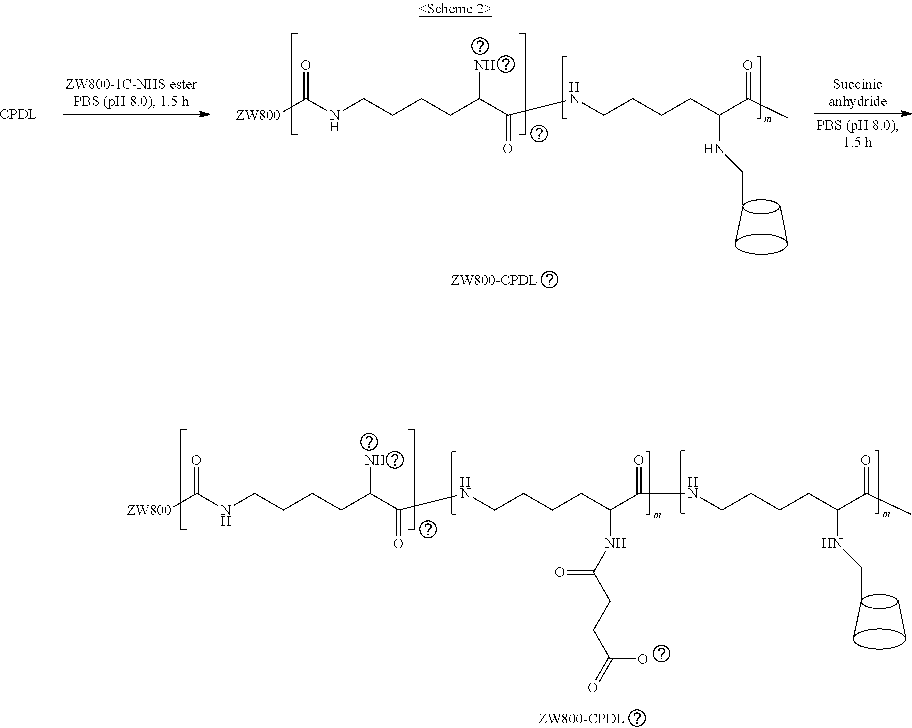

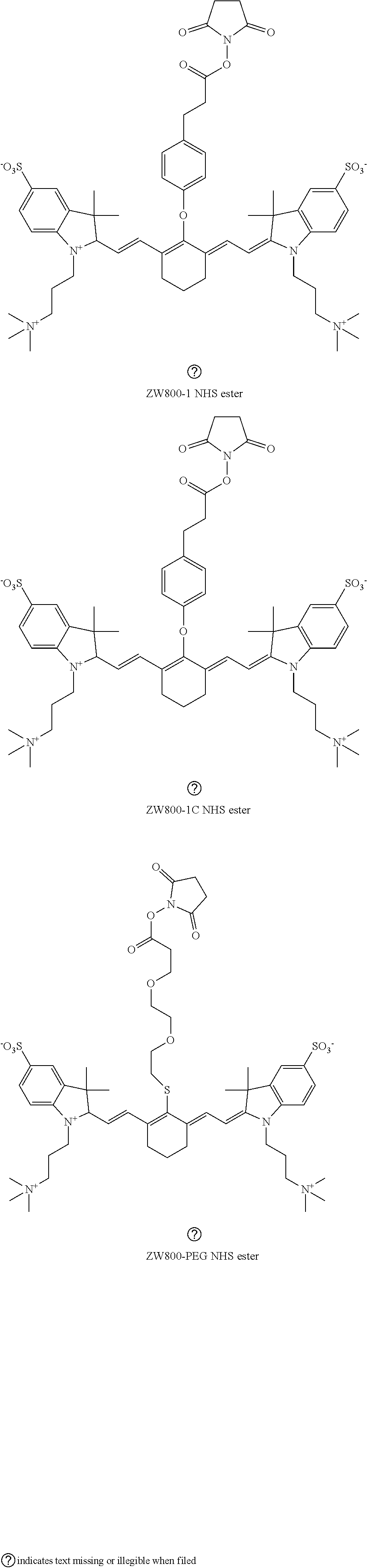

Furthermore, the present invention provides a composition for treating gout, the composition containing the cyclodextrin-conjugated polymer nano-drug produced by the preparation method, and the present invention has the following technical effects.

The nano-drug of the present invention may be administered through various routes (e.g., intravenous, intranasal, intradermal, or oral administration of a pharmaceutical composition form). These compositions may be prepared as described herein or otherwise and may be administered by various routes. The administration route depends on whether topical or systemic treatment is needed or on the area to be treated. In some embodiments, the administration may be parenterally performed. Examples of parenteral administration include intravenous, intraarterial, subcutaneous, intraperitoneal, or intramuscular administration. The parenteral administration may be performed in the form of a single bolus dose. The nano-drug provided in the present invention is suitable for intravenous administration. Conventional pharmaceutical carriers, aqueous, powdery, or oily bases, thickeners, and the like may be essential or desirable.

The nano-drug including a therapeutic agent of the present invention is combined with at least one pharmaceutically acceptable carrier (e.g., an excipient). In the preparation of the composition provided herein, an active ingredient is typically mixed with an excipient. For example, the active ingredient may be mixed or encapsulated with an excipient within a corresponding carrier in a capsule or tablet form or in other containers.

The pharmaceutical composition containing the nano-drug of the present invention may be in the form of tablets, pills, powders, suspensions, emulsions, solutions, syrups, aerosols (as solid or liquid media), soft and hard gelatin capsules, suppositories, sterile injectable solutions, and sterile packaged powders.

Some examples of the excipient suitable for the nano-drug of the present invention may be selected from lacto sugars, dextrose, sucrose, sorbitol, mannitol, starch, gum acacia, calcium phosphate, alginate, tragacanth, gelatin, calcium silicate, microcrystalline cellulose, polyvinylpyrrolidone, cellulose, water, syrups, methyl cellulose, and the like. The agent may further include, without limitation, lubricants such as talc, magnesium stearate, and mineral oils, wetting agents, emulsifying and suspending agents, preservatives such as methyl and propyl hydroxy-benzoates, sweetening agents, flavoring agents, or combinations thereof.

The nano-drug of the invention (e.g., a nano-drug containing at least one therapeutic agent) may be effective over a wide area of administration and is generally administered at a pharmaceutically effective amount. However, the dose of the nano-drug (e.g., a nano-carrier containing at least one pharmaceutical agent) is understood to be determined by a physician, and selected according to the administration route including the conditions being treated, the actual compound administered, the age, weight, and response of a subject, and the severity of symptoms of the subject.

Examples of an excipient and a diluent, which are carriers that may be contained in the pharmaceutical composition of the present invention, may include lactose, dextrose, sucrose, sorbitol, mannitol, xylitol, erythritol, maltitol, starch, gum arabic, alginate, gelatin, calcium phosphate, calcium silicate, cellulose, methyl cellulose, microcrystalline cellulose, polyvinyl pyrrolidone, water, methyl paraoxybenzoate, propyl paraoxybenzoate, talc, magnesium stearate, and mineral oils, but are not limited thereto. The pharmaceutical composition may be formulated as a preparation by using a typically used diluent or excipient, such as a filler, a stabilizer, a binder, a disintegrant, or a surfactant. Examples of a solid preparation for oral administration include tablets, pills, powders, granules, capsules, and the like. Such solid preparations may be prepared by mixing the compound of the present invention with at least one excipient, for example, starch, microcrystalline cellulose, sucrose or lactose, low-substituted hydroxypropyl cellulose, hypromellose, or the like. Also, lubricants, such as magnesium stearate and talc, may be used in addition to the simple excipients. Examples of a liquid preparation for oral administration correspond to suspensions, oral solutions, emulsions, syrups, and the like, and may include not only simple diluents that are frequently used, such as water and liquid paraffin, but also several types of excipients, for example, wetting agents, sweetening agents, flavoring agents, and preservatives.

Examples of a preparation for parenteral administration include sterile aqueous solutions, non-aqueous solvents, suspensions, emulsions, freeze-dried preparations, and suppositories. As the non-aqueous solvents and suspensions, propylene glycol, polyethylene glycol, a vegetable oil such as an olive oil, an injectable ester such as ethylolate, and the like may be used. As substrates for the suppositories, Witepsol, Macrogol, Tween 61, cacao butter, laurin butter, glycerol, gelatin, and the like may be used. For the formulation into a dosage form for parenteral administration, the nano-drug may be sterilized or mixed with an adjuvant, such as a preservative, a stabilizer, a hydrator, an emulsion promoter, a salt for regulation of osmotic pressure, or a buffer, and other therapeutically useful substances in water to be prepared into a solution or a suspension, which is then formulated in an ample or a vial unit dosage form.

The pharmaceutical composition containing the nano-drug disclosed in the present invention may be administered to mammalian animals, such as mice, livestock, and humans, via various routes. All modes of administration are possible, and for example, the administration may be performed orally, rectally, or by intravenous, intramuscular, subcutaneous, intrauterine dural, or intracerebroventricular injection. The dose may vary depending on the age, sex, or body weight of a subject to be treated, the particular disease or pathological condition to be treated, the severity of the disease or pathological condition, the time of administration, the route of administration, the absorption, distribution, and excretion rate of a drug, the kind of another drug used, the determination of a prescriber, and the like. The determination of the dose based on these factors is within the level of a person skilled in the art, and the general dose is in the range of approximately 0.01 mg/kg/day to approximately 2000 mg/kg/day. A more preferable dose is 1 mg/kg/day to 500 mg/kg/day. The administration may be performed once a day or divided into multiple doses. The dose is not intended to limit the scope of the present invention in any aspect.

### Advantageous Effects of Invention

The present invention can provide an excellent composition for treating gout, the composition containing a nano-drug in which a cyclodextrin moiety is conjugated to an amino group of a polymer.

The present invention can provide an excellent composition for treating gout or a method for treating gout, the composition containing a nano-drug in which a cyclodextrin moiety is conjugated to an amino group of a polymer, wherein a visible or near-infrared fluorophore is further conjugated to a carboxyl group of the polymer.

Furthermore, the present invention can provide an excellent composition for treating gout or a method for treating gout, the composition containing a nano-drug in which a cyclodextrin moiety is conjugated to an amino group of a polymer, wherein the nano-drug further contains at least one gout therapeutic agent, which forms a complex together with the cyclodextrin moiety.

### BRIEF DESCRIPTION OF DRAWINGS

For illustrating technical solutions in exemplary embodiments of the present invention or in the prior art more clearly, drawings required for use in the description of the exemplary embodiments will be introduced briefly below. It would be obvious that the drawings in the following description are provided as some exemplary embodiments of the present invention, for those of ordinary skilled in the art.

FIG. 1A schematically shows the structure of a cyclodextrin-conjugated polymer nano-drug of the present invention; FIG. 1B schematically shows an NIR fluorophore-cyclodextrin-conjugated EPL (NIR fluorophore-CDPL^{±}, NIR fluorophore-cyclodextrin ε-poly-L-lysine); and FIG. 1C shows the structure of fluorophore ZW800-cyclodextrin-conjugated EPL (ZW800-CDPL^{±}, cyclodextrin ε-poly-L-lysine).

FIG. 2 shows the results confirming the degree of binding of cyclodextrin of the present invention to uric acid (UA).

FIG. 3 shows an in vitro test for examining the uric acid crystal removal effect of ZW800-CDPL^{±}.

FIG. 4 shows an animal test protocol of a mouse gout model for examining the gout treatment effect of cyclodextrin-conjugated EPL (CDPL^{±}).

FIG. 5 shows the mouse gout model test results for a cyclodextrin-conjugated EPL (CDPL^{±}) administration group of the present invention and a saline administration group, wherein the mouse tophi size was compared by using a KFLARE imaging analyzer in the cyclodextrin-conjugated EPL (CDPL^{±}) administration group of the present invention and the saline administration group.

FIG. 6 shows the mouse gout model test results for a cyclodextrin-conjugated EPL (CDPL^{±}) administration group of the present invention and a saline administration group, wherein graphs shows the comparison of tophi size changes for 14 days in each group (n=3).

FIG. 7 shows the comparison of weight changes for 14 days in the mouse gout model test for a cyclodextrin-conjugated EPL (CDPL^{±}) administration group of the present invention and a saline administration group.

### BEST MODE FOR CARRYING OUT THE INVENTION

To illustrate various exemplary embodiments of the present invention in detail, the detailed description should not be considered as a limitation to the present invention but should be understood as the more detailed description of some certain aspects, features, and technical schemes of the present invention.

Unless otherwise indicated, all of technical and scientific terms used herein have the same meaning as commonly understood by those skilled in the art. Although the present invention illustrates only preferable methods and materials, any method and material similar or equivalent to those described herein can also be used in the implementation or testing of the present invention. All of the literature referred to herein are incorporated by reference for the purpose of disclosing and describing the method and/or materials associated with the literature.

### Example 1: Preparation of NIR Fluorophore-CDPL^{±}

### 1-1. Preparation of Aldehyde-Cyclodextrin (Ald-CD)

After β-cyclodextrin (15 g, 13.22 mmol) was dissolved in 100 mL of anhydrous DMSO, DMP (6.73 g, 15.86 mmol) was added to the resultant solution, followed by stirring at room temperature for 2 hours. After 2 hours, the reaction solution was poured into an ethyl acetate/acetone (20% v/v) mixture at 4° C. and stored at 4° C. overnight. The next day, the precipitate was collected by vacuum filtration and dissolved in DMSO, and then this solution was poured into cool ethyl acetate/acetone (20% v/v), followed by centrifugation at 3000 rpm for 15 minutes, and the supernatant was discarded. The precipitate was dissolved in deionized water and again poured into ethyl acetate/acetone to form a precipitate. The precipitate was dissolved in 75 mL of deionized water, stirred for 1 hour, and then freeze-dried to remove acetone and DMSO solvent. After the freeze-drying, 16 g of Ald-CD as a white solid was obtained.

### 1-2. Binding of EPL to Ald-CD

Ald-CD (16 g, 14.10 mmol) was dissolved in 550 mL of an acetic acid buffer (0.2 M, pH 4.5), and then EPL (2.2 g, 0.564 mmol) was added. After stirring at room temperature for 2 hours, the reaction mixture was reduced to an amine by addition of sodium borohydride (1 g, 26.45 mmol), and then continuously stirred at room temperature for 72 hours, followed by dynamic dialysis using a cellulose film with a molecular weight cut-off value of 6-8 kDa for 24 hours. After the dialysis, the resultant material was freeze-dried at −80° C. to obtain 3.75 g of an off-white solid. For characterization of β-CD-conjugated EPL (CDPL), the number of β-CD moieties conjugated to EPL was determined using 1H-NMR spectrometry. In addition, size exclusion chromatography (SEC) was used to determine the purity of CDPL and confirmed the absence of un-conjugated Ald-CD.

### 1-3. Binding of NIR Fluorophore and CDPL (ZW800-CDPL^{±} Preparation)

ZW800-1C (500 mg, 0.5 mmol) was dissolved in 50 mL of anhydrous DMSO, and then 0.5 mL of N,N-diisopropylethylamine (DIEA) and dipyrrolidino (N-succinimidyloxy) carbenium hexafluorophosphate (HSPyU) (410 mg, 1 mmol) were added, followed by stirring at room temperature for 2 hour. Thereafter, the reaction mixture was added to 250 mL of acetone/ethanol (1:1 v/v), and then the resultant precipitate was filtered, and washed with acetone/ethanol to remove excessive reagents. Then, the activated fluorophore ZW800-1C NHS ester was dried under vacuum and produced.

The ZW800-1C NHS ester (50 μmol) was added to a solution in which CDPL (400 mg, 25 μmol) was dissolved in 5 ml of PBS (pH 8.0), and then the reaction mixture was stirred at room temperature for 12 hours. Excessive reagents were removed by a centrifuge (10 kDa MWCO), and the filtrate was freeze-dried to prepare ZW800-CDPL^{±} having a positive charge.

1-4. Succinylation of ZW800-CDPL^{±} (ZW800-CDPL^{±} Preparation)

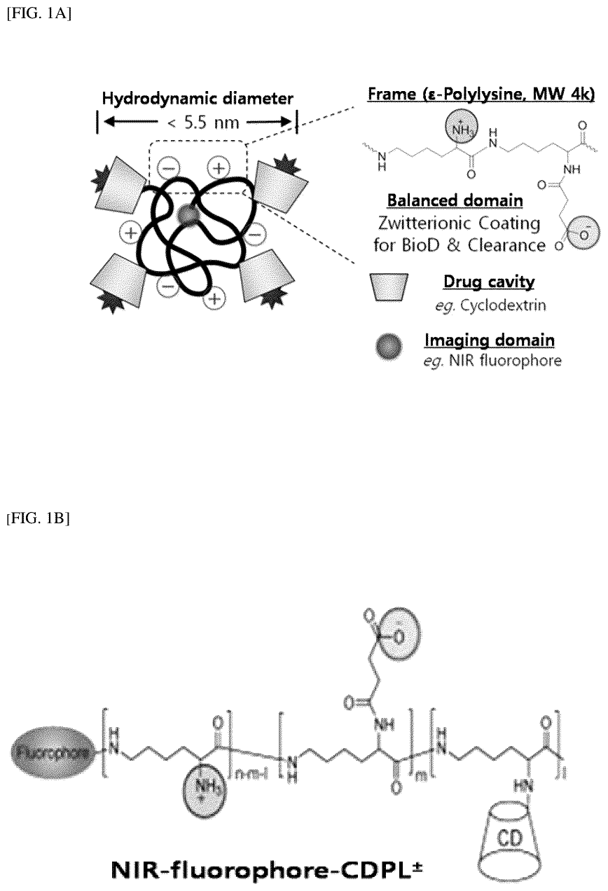

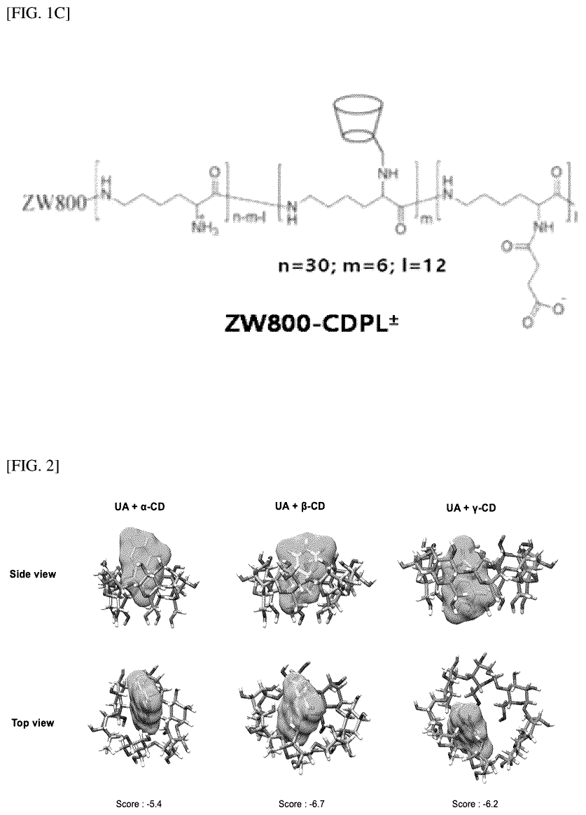

To obtain zwitterionic ZW800-CDPL^{±} (having both positive and negative ions), the ZW800-1C-NHS ester (50 μmol) was added to CDPL (400 mg, 25 μmol) in 5 mL of PBS (pH 8.0). The reaction mixture was stirred for 12 hours, then succinic anhydride (SA; 3 n μmol, n is the number of lysine units in EPL) was added to the reaction mixture, and the mixture was stirred at room temperature for 1.5 hours. The solution was subjected to precipitation by addition of acetone (14 mL) and washed five times with acetone, and then excessive reagents were removed by centrifugation. The resultant filtrate was freeze-dried to prepare ZW800-CDPL^{±} having both positive and negative charges. (see FIG. 1).

### Test Example 1: Uric Acid Removal Effect in In Vitro Test of ZW800-CDPL^{±}

After ZW800-CDPL^{±} prepared in Example 1 was dissolved in PBS to prepare a stock solution (1 mM), a membrane chamber (molecular weight cut-off (MWCO): 3.5 K) was weighed, and then 2 mg of uric acid crystals were placed in the upper chamber, and 1.2 mL of the ZW800-CDPL^{±} solution or PBS solution was placed into Eppendorf tubes (EP tubes). In addition, the tubes were shaken at 20 rpm at 37° C. by an up-and-down shaker, and then 1 mL of a release solution was taken from each tube by a pipette at the same time. Thereafter, 1 mL of a fresh buffer was returned at the same temperature and at the same time, and then the uric acid crystals remaining in the membrane chamber were weighed, and the weight measurement results are shown in Table 1.

TABLE 1

| | | ZW800-CDPL^{±} treated |
|---|---|---|
| | Comparison group (PBS) | group |
| Uric acid residual | 1.7 ± 0.29 | 0.43 ± 0.25 |
| amount | | |

As shown in Table 1, the uric acid removal (entrapment) effect of the ZW800-CDPL±-treated group was excellent by approximately 4 times compared with the comparison group (PBS).

### Test Example 2: Gout Treatment Effect of ZW800-CDPL^{±} in Animal Model

### 2-1. Animal Test Method

Test animal preparation: After the furs of mice were removed, monosodium urate (MSU, 10 mg/mouse) was subcutaneously injected to the back of the mice (N=3).
Test groups: saline (n=3), ZW800-CDPL^{±} (n=3)
Injection concentration: ZW800-CDPL^{±} (20 mg/mouse/day), potassium oxonate (300 mg/Kg/day)
Administration route: subcutaneous injection (ZW800-CDPL^{±}, saline), intraperitoneal injection (PO)
Drug administration method: intraperitoneal injection of PO was conducted daily until D14 from one day before MSU injection; subcutaneous injection of ZW800-CDPL^{±} or saline was conducted daily until D14 from four days after MSU injection; and the test was ended on D15.
Evaluation method: Tophi size, KFLARE imaging analysis
Tophi size: Size measurement (V=½*LW²; V=Volume, L=Length, W=Width)
Imaging: Taken daily until D15 before ZW800-CDPL^{±} injection

| | Molecular | | | | |
|---|---|---|---|---|---|
| | weight | | Reconstitution | Injection | Injection |
| Sample | (MW) | Amount | volume | dose | volume |
| CDPL^{±} | 14050 | 60 mg/ | 300 μL/Tube | 50 μmol/kg | 100 μL |
| | | Tube × | | | |
| | | 14 tubes | | | |

Total amount: 840 mg

The animal test protocol of the mouse gout model for examining the gout treatment effect of cyclodextrin-conjugated EPL (ZW800-CDPL^{±}) of the present invention is summarized in FIG. 4.

### 2-2. Animal Test Results

A test of comparing the tophi size in the cyclodextrin-conjugated EPL (ZW800-CDPL^{±}) administration group of the present invention and the saline administration group was conducted by using the KFLARE imaging analyzer, and as a result, the cyclodextrin-conjugated EPL (ZW800-CDPL^{±}) administration group showed an excellent tophi size reducing effect (see FIGS. 5 and 6). In a mouse gout model test for the cyclodextrin-conjugated EPL (ZW800-CDPL^{±}) administration group of the present invention, no weight change was observed for 14 days, indicating no toxicity (see FIG. 7).

### Preparation Example 1: Pharmaceutical Agents

### 1-1. Tablet Preparation

The ZW800-CDPL^{±} nano-drug (100 mg) of the present invention was mixed with 175.9 g of lactose, 180 g of potato starch, and 32 g of colloidal silicate. After a 10% gelatin solution was added to this mixture, the resultant solution was milled and passed through a 14-mesh sieve. The resultant product was dried, and 160 g of potato starch, 50 g of an active ingredient, and 5 g of magnesium stearate were added thereto, thereby obtaining a mixture, which was then prepared into tablets.

### 1-2. Injection Preparation

The ZW800-CDPL^{±} nano-drug (100 mg) of the present invention, 0.6 g of sodium chloride, and 0.1 g of ascorbic acid were dissolved in 0.1 g of distilled water to prepare 100 mL of a solution. This solution was placed in a bottle, and sterilized by heating at 120° C. for 30 minutes.

1. A pharmaceutical composition for treating gout, the pharmaceutical composition comprising a nano-drug in which a cyclodextrin moiety is conjugated to an amino group of a polymer, wherein the nano-drug contains an average of 5 to 14 cyclodextrin moieties and has an average molecular weight of about 10,000 to 22,000 g/mol.

2. The pharmaceutical composition of claim 1, wherein the nano-drug has an overall positive charge, an overall negative charge, or no charge.

3. The pharmaceutical composition of claim 1, wherein the cyclodextrin moiety is selected from β-cyclodextrin, γ-cyclodextrin, 2-hydroxypropyl-β-cyclodextrin, 2-hydroxypropyl-γ-cyclodextrin, methyl-β-cyclodextrin, β-cyclodextrin thioether, or cyanoethylated β-cyclodextrin.

4. The pharmaceutical composition of claim 1, wherein the polymer is selected from ε-poly-L-lysine, L-polylysine, polylactic acid, poly(lactic-co-glycolic acid), polyaspartic acid, polyglutamic acid, or polyglutamic acid-poly(ethylene glycol) copolymers.

5. The pharmaceutical composition of claim 1, wherein in the nano-drug, a fluorophore is further conjugated to a carboxyl group of the polymer, the fluorophore being selected from a clinically approved visible fluorophore a near-infrared fluorophore under a clinical trial, or an other near-infrared fluorophore.

6. The pharmaceutical composition of claim 1, further comprising a gout therapeutic agent, which forms a complex together with the cyclodextrin moiety.

7. The pharmaceutical composition of claim 6, wherein the gout therapeutic agent is selected from aceclofenac, loxoprofen, meloxicam, lornoxicam, naproxen+esomeprazole, etodolac, nabumetone, indometacin, diclofenac, naproxen, zaltoprofen, dexibuprofen, pelubiprofen, ibuprofen, talniflumac acid, morniflumate, aspirin, ketoprofen, piroxicam, mefenamic acid, COX-2 selective inhibitor, celebrex, opioid receptor agonist, tramadol, colchicine, hydrocortisone, dexamethasone, methylprednisolone, benzburomarone, xanthine oxidase inhibitor, allopurinol, or febuxostat.

8. The pharmaceutical composition of claim 5, wherein the clinically approved visible fluorophore is selected from fluorescein, Cy3, Cy5, Cy5.5, Cy7, Cy7.5, or Oxazine.

9. The pharmaceutical composition of claim 5, wherein the near-infrared fluorophore under a clinical trial is selected from ZW800-1, ZW800-1C, ZW800-3C, ZW800-PEG, ZW700-1, indocyanine green (ICG), IRDye800-CW (CW800), or OTL38.

10. A method for treating gout, comprising administering to a mammal in need thereof an effective amount of a nano-drug containing a gout therapeutic agent, in which a cyclodextrin moiety is conjugated to an amino group of a polymer, wherein the nano-drug contains an average of 5 to 14 cyclodextrin moieties and has an average molecular weight of about 10,000 to 22,000 g/mol and wherein the gout therapeutic agent forms a complex together with the cyclodextrin moiety.

11. The method of claim 10, wherein the nano-drug has an overall positive charge, an overall negative charge, or no charge.

12. The method of claim 10, wherein the cyclodextrin moiety is selected from β-cyclodextrin, γ-cyclodextrin, 2-hydroxypropyl-β-cyclodextrin, 2-hydroxypropyl-γ-cyclodextrin, methyl-β-cyclodextrin, β-cyclodextrin thioether, or cyanoethylated β-cyclodextrin.

13. The method of claim 10, wherein the polymer is selected from ε-poly-L-lysine, L-polylysine, polylactic acid, poly(lactic-co-glycolic acid), polyaspartic acid, polyglutamic acid, or polyglutamic acid-poly(ethylene glycol) copolymers.

14. The method of claim 10, wherein in the nano-drug, a fluorophore is further conjugated to a carboxyl group of the polymer, the fluorophore being selected from a clinically approved visible fluorophorea near-infrared fluorophore under a clinical trial, or an other near-infrared fluorophore.

15. The method of claim 10, wherein the gout therapeutic agent is selected from aceclofenac, loxoprofen, meloxicam, lornoxicam, naproxen+esomeprazole, etodolac, nabumetone, indometacin, diclofenac, naproxen, zaltoprofen, dexibuprofen, pelubiprofen, ibuprofen, talniflumac acid, morniflumate, aspirin, ketoprofen, piroxicam, mefenamic acid, COX-2 selective inhibitor, celebrex, opioid receptor agonist, tramadol, colchicine, hydrocortisone, dexamethasone, methylprednisolone, benzburomarone, xanthine oxidase inhibitor, allopurinol, or febuxostat.
